(19) United States
(12) Patent Application Publication
(10) Pub. No.: US 2013/0280231 A1
(43) Pub. Date: Oct. 24, 2013

(54) COMPOSITION FOR PREVENTING OR TREATING OBESITY

(71) Applicant: ELE LIFE SCIENCE CO., LTD., Gwangju (KR)

(72) Inventor: Jae Cheon LEE, Gwangju (KR)

(73) Assignee: ELE Life Science Co., Ltd., Gwangju (KR)

(21) Appl. No.: 13/866,614

(22) Filed: Apr. 19, 2013

(30) Foreign Application Priority Data
Apr. 20, 2012 (KR) 10-2012-0041595

(51) Int. Cl. A61K 38/22; A61K 38/28; A61K 38/51; A61K 45/06

(52) U.S. Cl. CPC A61K 38/22; A61K 45/06; A61K 38/28; A61K 38/51
USPC 424/94.5; 514/4.8

(57) ABSTRACT

Provided is a composition for preventing or treating obesity, and more particularly, a composition for preventing or treating obesity in order to treat or improve states of obese patients requiring weight control. The composition according to the present invention has advantages in that side effects by administration of the composition are not generated, and effects of losing weight and maintaining weight are excellent without changing food intake and consumption amounts, such that the composition may be effectively used to prevent overweight and obesity related diseases.

### CROSS-REFERENCE TO RELATED APPLICATIONS

This application claims priority under 35 U.S.C. §119 to Korean Patent Application No. 10-2012-0041595, filed on Apr. 20, 2012, in the Korean Intellectual Property Office, the disclosure of which is incorporated herein by reference in its entirety.

### TECHNICAL FIELD

The following disclosure relates to a composition for preventing or treating obesity, and more particularly, to a composition for preventing or treating obesity in order to treat or improve states of obese patients requiring weight control.

### BACKGROUND

Obesity, which is one of the common nutritional disorders in the world, indicates a phenomenon that extra calories are accumulated in a form of fat in the body by eating more calories than consumed calories. According to the statistics of World Health Organization (WHO), currently, about 250 million people are classified as obese patients, and it is expected that about 300 million people will suffer from obesity after 20 years. About 50% of the adults in the United States are considered overweight or obese with a body mass index (BMI) of 25 or more, and it has been reported that one in four adults are obese with a BMI of 25 or more according to the domestic statistics (Ministry of Health and Welfare. Report on 1998 National Health and Nutrition Survey, 2000; Lee B G., et al., J. Kor. Soc. Study Obesity, 11(2), pp. 131-140, 2002).

It was thought that obesity is induced by various causes such as a genetic influence, an environmental influence by westernized diet, psychological impact by stress, or the like, but exact causes or mechanism thereof were not clearly established yet. However, since obesity may act as a cause of diseases such as cardiovascular diseases or diabetes as well as problems of obesity itself, (Manson, et al., New England J. Med., 333, pp. 677-685, 1995; Kopleman P. G., Nature, 404, pp. 635-643, 2000; Must, et al., JAMA, 282, pp. 1523-1529, 1999), the interest in treatment of obesity has increased all over the world.

In addition, since obesity is strongly involved in incidence of various adult diseases, as well as an external problem, the higher the degree of obesity, the higher the prevalence rates of diabetes, cholelithiasis, hypertension, a cardiac disorder, a stroke, or the like (Field A. E., et al., Arch. Intern, Med., 161(13), pp. 1581-1586, 2001; Cha B. R., et al., Kor. J. Nutr., 36(5), pp. 483-490, 2003). Accordingly, the financial burden and a loss of lives caused by obesity are enormous. According to the results of the epidemiological survey that was performed in the US, the number of deaths due to diseases related to obesity reached 28,000 in 1991, approximately 80% of them had morbid obesity with a BMI of 30 or more, and approximately 120 trillion won were spent for treating the obesity-related diseases in a year.

In accordance with the growing interest in regulating body weight as described above, it was reported that 48.1% of women in their 20's and 40% of women in their 30's had the experience in losing weight in the past year (Kae S. H., Patterns of Body Weight and Diet for Korean-1998 National Health and Nutrition Survey—Proceeding for Korean Community Nutrition., Society Spring Conference, 7-28, 2001). The domestic weight loss market has grown rapidly, such that the market was estimated to be 200 billion won in 2001 and 300 billion won in 2002. Further, it was announced that weight-control foods of which importation at normal prices was approved were increased 110 times for the past 2 years. However, effects of these products are doubtful, and a lot of side effects have been reported during the usage time (Lee B G., et al., J. Kor. Soc. Study Obesity, 11(2), pp. 131-140, 2002; Food and Environment, Sep. 18, 2002).

Recently, it was reported that our obese population has increased by 1.6-fold over the past 10 years, and as shown by the developed countries as any guide, the obese population will be more rapidly increased in the future (in a press release of the Ministry of Health and Welfare, 2006).

It is known that obesity increases the incidence of adult diseases or chronic degenerative diseases, such that medical cost of obesity related diseases occupies a high percentage of the entire medical cost. In the case of developed countries, 2 to 7% of the overall national medical cost consumed is due to overweight and obesity, and it is estimated that when indirect cost such as cost involved in informal medicine service, diet food, exercise, degradation of productivity due to obesity, and the like, and intangible factors such as degradation of the quality of life, pain caused by disease, and the like, are included, social and economic cost due to obesity will occupy a higher percentage.

Various method and studies for treating obesity have been suggested, and it has emphasized that since extra energy is accumulated in the body due to excessive intake of energy or a decrease of consumed energy to cause obesity, in order to treat obesity and maintain an appropriate weight, it is particularly important to lose weight in addition to dieting.

Only when fat accumulated in the body is decreased due to a decrease in caloric intake and an increase in calorie consumption, weight loss may be effectively achieved. That is, the development of a practical method capable of regulating food intake to lose weight while appropriately getting all the nutrients with the aid of a functional food or a functional supplement to help the treatment of obesity without any side effect has been demanded.

Therefore, while conducting continuous studies in order to develop a diet product as described above, the present inventors confirmed that in the case in which a mixture of leptin based polypeptide including insulin; and an adenosine 5′-monophosphate-activated protein kinase (AMPK) activator was injected, an amazing weight loss effect and durability of the effect were maintained, thereby completing the present invention.

### RELATED ART DOCUMENT

### Patent Document

(Patent Document 1) KR 10-2006-0014629 A (Feb. 16, 2006)

### SUMMARY

An embodiment of the present invention is directed to providing a composition for preventing or treating obesity containing leptin based polypeptide; an adenosine 5′-monophosphate-activated protein kinase (AMPK) activator; and insulin as active ingredients.

In one general aspect, a composition for preventing or treating obesity contains leptin based polypeptide; an adenosine 5′-monophosphate-activated protein kinase (AMPK) activator; and insulin as active ingredients.

The composition according to the present invention is characterized by using a mixture of the leptin based polypeptide; the adenosine 5′-monophosphate-activated protein kinase (AMPK) activator; and insulin as the active ingredients.

Hereinafter, the present invention will be described in detail.

As used herein, the term “obesity” may include diseases selected from a group consisting of overeating, overdrinking, hyperphagia, hypertension, diabetes, increased plasma insulin concentrations, insulin resistance, hyperlipidemia, metabolic syndrome, insulin resistance syndrome, obesity-related gastro-esophageal reflux disease (GERD), arteriosclerosis, hypercholesterolemia, hyperuricemia, lower back pain, cardiac hypertrophy and left ventricular hypertrophy, lipodystrophy, non-alcoholic steatohepatitis, cardiovascular disease, polycystic ovary syndrome, and people wanting to lose weight may also be included to the target for treating these diseases.

In addition, as used herein, the term “obesity” is generally defined as the case in which a body mass index (BMI) is more than 30, but in order to achieve the object of the present invention, all the people requiring weight loss or prevention of weight gain or wanting to lose weight even though their BMI's are less than 30 are also included in a category of “obese”. Therefore, people having a BMI less than 30, people having a BMI of 25 or more (considered as overweight), or people having a BMI less than 25 may be included in the target of the present invention. Morbid obesity indicates the case in which a BMI is 40 or more.

As used herein, the term “leptin based polypeptide” means a peptide that has a slightly different kind of amino acid from that of leptin protein but has a structure or function similar to those of the leptin protein, and the polypeptide as described above may include leptin or leptin derivatives.

Leptin is a small protein (167 amino acid residues) made by adipose cells, moving through bloods, acting on a receptor of hypothalamus in brain to decrease appetite. A leptin-receptor interaction in the hypothalamus changes secretion of neurotransmitter affecting the appetite. Leptin also stimulates a sympathetic nervous system to increase blood pressure, a heart rate, thermogenesis by uncoupling electron transport from synthesis of ATP in the mitochondria of adipose cells. In addition, leptin stimulates generation of appetite suppressing peptide hormone and induces signal transduction cascade for regulating gene expression.

In this case, the leptin derivative has an identity of at least 70% (for example, 80%, 90%, 95%, 100%, or a different number of 70 to 100%) with respect to the amino acid sequence of leptin and substantially has activities of leptin such as activity of binding to the receptor thereof and promoting signal transduction pathway corresponding thereto, and the like.

“% identity” between two amino acid sequences or two nucleic acids is determined using an algorithm by Karlin and Altschul Proc. Natl. Acad. Sci. USA 87:2264-68 (1990), modified by Karlin and Altschul Proc. Natl. Acad. Sci. USA 90:5873-77 (1993). The algorithm as described above is incorporated into the NBLAST and XBLAST programs (version 2.0) by Altschul, et al. J. Mol. Biol. 215:403-10 (1990). BLAST nucleotide searches may be performed with the NBLAST program, score=100, wordlength=12, in order to obtain nucleotide sequences homologous to nucleic acid molecules of the present invention. BLAST protein searches may be performed with the XBLAST program, score=50, wordlength=3, in order to obtain amino acid sequences homologous to protein molecules of the present invention. Where gaps exist between two sequences, gapped BLAST may be utilized as described in Altschul et al., Nucleic Acids Res., 25(17):3389-3402 (1997). When utilizing BLAST and gapped BLAST programs, the default parameters of the respective programs (for example, XBLAST and NBLAST) may be used.

As used herein, the term “AMPK activator” means an agent activating adenosine 5′ -monophosphate-activated protein kinase (AMPK) to phosphorylate its substrates, and Examples of the AMPK activator may include acetyl-CoA carboxylase and malonyl-CoA decarboxylase.

In detail, as the AMPK activator, at least one selected from adiponectin, metformin, phenformin, buformin, thienopyridone, 5′-aminoimidazole-4-carboxamide ribonucleoside (AICAR), resveratrol, nootkatone, and thiazoles may be used, and among them adiponectin may be preferably used.

The adiponectin, which is a peptide hormone (224 amino acid residues), is produced in almost only adipose tissue. The adiponectin circulates in blood and strongly affects metabolisms of fatty acid and carbohydrate in liver or muscle. The adiponectin increases fatty acid influx from blood into muscle cells and a fatty acid oxidation rate in the muscle. Further, the adiponectin inhibit synthesis of fatty acid and glucose in liver cells and stimulates sugar influx into muscle and liver and anabolism These actions of adiponectin is indirectly performed through the AMPK, which is a key regulatory enzyme activated by an increase of intracellular AMP, and the AMPK also regulates beta oxidation of fatty acids.

As used herein, the term “insulin” may include insulin from various sources such as recombinant DNA and animal sources as well as human insulin, porcine insulin, bovine insulin, bovine-porcine insulin, regular insulin, neutral protamine hagedorn (NPH) and LENTS® type insulin. Other examples of the insulin may include mixtures of insulin (for example, NPH, human, and porcine insulin) in various forms. Other examples of insulin may include insulin mixed with other ingredients such as zinc crystals or in a phosphate buffer as well as mixtures of Insulin Lispro Protamine and Insulin Injection (rDNA origin), a mixture of 50/50 (or a 70/30) Human Insulin Isophane Suspension and Human Insulin Injection, a mixture of 70/30 NPH Human Insulin Isophane Suspension and Human Insulin Injection (rDNA), insulin glargine, insulin lispro, insulin aspart. Insulin may also be obtained from Saccharomyces cerevisiae or other sources.

In the present invention, the composition may contain 1 to 300 ng/ml of the leptin based polypeptide, 1 to 200 μg/ml of the AMPK activator, and 0.1 to 30 ng/ml of insulin as the active ingredients. More preferably, the composition may contain 25 to 250 ng/ml of the leptin based polypeptide, 50 to 150 μg/ml of the AMPK activator, and 1 to 15 ng/ml of insulin as the active ingredients.

More specifically, in the case in which leptin, adiponectin, and insulin each were individually contained or a mixture of two of them was contained in the composition, initially, weight slightly decreased but increased again. However, it was confirmed that significant weight loss effect was achieved and durability of the effect was maintained by using the mixture of leptin, adiponectin, and insulin.

The composition according to the present invention may further contain pharmaceutically acceptable salts.

As used herein, the term “pharmaceutically acceptable salts” means compound salts that do not cause significant irritation in an organism at the time of administration and destroy biological activities and properties of the compound. Pharmaceutically acceptable salts may be obtained by reacting the compound according to the present invention with inorganic acid such as hydrochloric acid, bromic acid, sulfuric acid, nitric acid, phosphoric acid, methane sulfonic acid, ethane sulfonic acid, p-toluene sulfonic acid, salicylic acid, or the like. The pharmaceutically acceptable salts may be obtained by reacting the compound according to the present invention with base so as to form ammonium salts, alkali metal salts such as sodium or potassium salts, alkali earth metal salts such as calcium or magnesium salts, salts of organic bases such as dicyclohexylamine, N-methyl-D-glucamine, tris(hydroxymethyl)methylamine, and salts with amino acids such as arginine, lysine, and the like.

In another general aspect, the present invention may be used in a method of treating obesity related disease, a method of suppressing food intake, and a method of losing body fat, using the composition as described above. More specifically, the method may include administering a therapeutically acceptable amount of composition.

As used herein, the term “administration” means that a predetermined material is introduced to the patient by some appropriate method, and the composition may be administered through any route as long as the route is a general route through which a drug may arrive at a target tissue. Examples of the administration include intraperitoneal administration, intravenous administration, intramuscular administration, subcutaneous administration, intradermal administration, oral administration, topical administration, intranasal administration, intrapulmonary administration, and rectal administration, but are not limited thereto. However, since peptides are digested at the time of oral administration, it is preferable that the composition for oral administration is coated with an active drug or formulated so as to be protected from degradation in the stomach. Preferably, the composition may be administered as an injection. In addition, the composition may be administered by an optional device capable of moving the active material to target cells. In this case, the therapeutically acceptable amount of the composition may be determined by various factors as described above.

As used herein, the term “patient” indicates a target to which the composition according to the present invention is administered, that is, an animal, preferably a mammal, and particularly, a person being in a progressive obese state or expected to have secondary metabolic diseases including diabetes, osteopenia, and osteoporosis due to obesity.

The composition for preventing or treating obesity according to the present invention having the weight loss effect as described above may further contain pharmaceutically or physiologically acceptable carriers, excipients, diluents, and the like except for the above-mentioned materials.

The composition according to the present invention may be formulated into oral type formulations such as powders, granules, tablets, capsules, suspensions, emulsions, syrups, aerosols, and the like, or sterile injection solutions by the general methods so as to correspond to using purposes thereof, respectively, to thereby be used. As the carrier, the excipient and the diluent that may be included in the composition, there are lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, mineral oil, and the like.

Solid formulations for oral administration include tablets, pills, powders, granules, capsules, and the like. The solid preparation as described above is formulated by mixing the composition with at least one excipient, for example, starch, calcium carbonate, sucrose, lactose, gelatin, or the like. Further, lubricants such as magnesium stearate or talc may be used in addition to simple excipients. Liquid formulations for oral administration include suspensions, solutions, emulsions, syrups, and the like, and various excipients, for example, a wetting agent, a sweetener, an aromatic piece, a preservant, or the like, as well as water and liquid paraffin that are generally used as simple diluents may be contained.

Formulations for parenteral administration include sterile aqueous solutions, non-aqueous solvents, suspensions, emulsions, freeze-dried formulations, and suppositories. In the non-aqueous solvent or the suspensions, propylene glycol, polyethylene glycol, vegetable oil such as olive oil, injectable esters such as ethyloleate, or the like, may be used. In a base for injection, existing additives such as a solubilizer, an isotonic agent, a suspending agent, an emulsifier, a stabilizer, and a preservative may be included.

An effective amount of the composition according to the present invention may be changed depending on the ages, the gender, and the weight of the patient. However, generally, the composition may be administered at a dose of 5 to 100 mg, preferably 10 to 50 mg, and more preferably 15 to 30 mg per kg body weight every day or every other day, or the dose may be divided into 1 to 3 times a day to thereby be administered. In addition, the administration dose may be increased or decreased according to the administration route, degree of disease, gender, weight, age, and the like. Therefore, the scope of the present invention is not limited to the administration dose.

The composition according to the present invention may be added to a functional health food for maintaining healthful life in addition to being used as a drug for preventing or treating obesity. As the health food to which the composition according to the present invention may be added, for example, there are various food, drink, gum, vitamin complexes, health assisting food, and the like.

The composition according to the present invention may be added to a food or a drink at a content of 0.001 to weight%, preferably 0.01 to 30 weight% of the total weight of the food. Particularly, the composition may be added to a health functional drink at a content of 0.01 to 20 g, preferably 0.1 to 10 g based on 100ml of the drink.

The health functional drink containing the composition according to the present invention may further contain various flavors generally added to drinks or natural carbohydrates as an additional ingredient in addition to the compound of claim 1. Examples of the natural carbohydrates include monosaccharide such as glucose, fructose, and the like, disaccharides such as maltose, sucrose, and the like, polysaccharides such as dextrin, cyclodextrin, and the like, and sugar alcohols such as xylitol, sorbitol, erythritol, and the like. As the flavor, natural flavors (stevia extracts such as rebaudioside A, glycyrrhizin, and the like, or thaumatin) and synthetic flavors (saccharine, aspartame, and the like) may advantageously be used. Besides the additional ingredients as described above, the composition according to the present invention may contain various nutrients, vitamins, minerals (electrolytes), flavorants such as synthetic flavorants and natural flavorants, colorants and fillers, pectic acid and salts thereof, alginic acid and salts thereof, organic acids, protective colloid thickeners, pH control agents, stabilizers, preservatives, glycerin, alcohols, carbonating agents used in a carbonated beverage, or the like. In addition, the composition according to the present invention may contain fruit fresh for preparing natural fruit juices, fruit juice beverages, and vegetable beverages. These ingredients may be used independently or in combination.

### DETAILED DESCRIPTION OF EMBODIMENTS

The present invention will be described in detail with reference to the following Examples. However, the following Examples are provided only for assisting in the understanding of the present invention, but the present invention is not limited to the following Examples.

Technical terms and scientific terms used in the present specification have the general meaning understood by those skilled in the art to which the present invention pertains unless otherwise defined, and a description for the known function and configuration obscuring the present invention will be omitted in the following description and the accompanying drawings.

### EXAMPLES AND COMPARATIVE EXAMPLES

Injections containing the composition shown in the following Table 1 were prepared by a general method for preparing an injection.

TABLE 1

| | Leptin | | | Adiponectin | | | Insulin | | |
|---|---|---|---|---|---|---|---|---|---|
| | (ng/m [symbol] ) | | | (μg/m [symbol] ) | | | (ng/m [symbol] ) | | |
| | 30 | 120 | 240 | 30 | 60 | 120 | 5 | 10 | 30 |
| Comparative | ○ | — | — | — | — | — | — | — | — |
| Example 1 | | | | | | | | | |
| Comparative | — | — | — | ○ | — | — | — | — | — |
| Example 2 | | | | | | | | | |
| Comparative | — | — | — | — | — | — | — | ○ | — |
| Example 3 | | | | | | | | | |
| Comparative | ○ | — | — | — | ○ | — | — | — | — |
| Example 4 | | | | | | | | | |
| Comparative | — | ○ | — | — | ○ | — | — | — | — |
| Example 5 | | | | | | | | | |
| Comparative | — | — | ○ | — | — | ○ | — | — | — |
| Example 6 | | | | | | | | | |
| Comparative | ○ | — | — | — | — | — | — | ○ | — |
| Example 7 | | | | | | | | | |
| Comparative | — | — | — | — | ○ | — | — | ○ | — |
| Example 8 | | | | | | | | | |
| Comparative | ○ | — | — | — | — | — | — | — | ○ |
| Example 9 | | | | | | | | | |
| Comparative | — | — | — | ○ | — | — | — | — | — |
| Example 10 | | | | | | | | | |
| Example 1 | ○ | — | — | ○ | — | — | ○ | — | — |
| Example 2 | — | ○ | — | — | ○ | — | — | ○ | — |
| Example 3 | — | — | ○ | — | — | ○ | — | — | ○ |
| Example 4 | — | ○ | — | ○ | — | — | — | ○ | — |
| Example 5 | — | — | ○ | — | — | ○ | ○ | — | — |

### EXPERIMENTAL EXAMPLE

### Measurement of Pharmacological Effect

(1) Change in Weight

After mice for the experiment were divided into 15 (Comparative Examples 1 to 10 and Examples 1 to 5) groups except for a not-treated group (control group), 500 0 of the injections prepared in Comparative Examples 1 to 10 and Examples 1 to 5 were intravenously administered to the 15 groups, respectively.

Change in weight (g) of the mice to which the injections prepared in Comparative Examples 1 to 10 and Examples 1 to 5 were administered, respectively were observed for 2 weeks.

TABLE 2

| | Experiment | | | | | | |
|---|---|---|---|---|---|---|---|
| | date | 1 | 3 | 5 | 7 | 10 | 14 |
| Not-treated | 274 | 273 | 275 | 275 | 275.5 | 275 | 275 |
| group | | | | | | | |
| Comparative | 283 | 275 | 274 | 276 | 278 | 280 | 285 |
| Example 1 | | | | | | | |
| Comparative | 303 | 293 | 292 | 292 | 295 | 297 | 299 |
| Example 2 | | | | | | | |
| Comparative | 302 | 302 | 305 | 305.5 | 306 | 307 | 308 |
| Example 3 | | | | | | | |
| Comparative | 398 | 392 | 380 | 370 | 368 | 375 | 383 |
| Example 4 | | | | | | | |
| Comparative | 395 | 390 | 375 | 371 | 366 | 376 | 385 |
| Example 5 | | | | | | | |
| Comparative | 378 | 371 | 365 | 362 | 368 | 371 | 373 |
| Example 6 | | | | | | | |
| Comparative | 311 | 302 | 297 | 292 | 295 | 302 | 309 |
| Example 7 | | | | | | | |
| Comparative | 344 | 341 | 330 | 325 | 320 | 338 | 342 |
| Example 8 | | | | | | | |
| Comparative | 302 | 282 | 276 | 272 | 284 | 289 | 297 |
| Example 9 | | | | | | | |
| Comparative | 294 | 283 | 275 | 269 | 278 | 283 | 291 |
| Example 10 | | | | | | | |
| Example 1 | 317 | 302 | 284 | 276 | 267 | 255 | 249 |
| Example 2 | 386 | 370 | 355 | 349 | 341 | 334 | 329 |
| Example 3 | 342 | 330 | 321 | 316 | 312 | 306 | 301 |
| Example 4 | 384 | 371 | 364 | 356 | 348 | 345 | 337 |
| Example 5 | 375 | 364 | 357 | 350 | 344 | 340 | 333 |

As shown in Table 2, it may be confirmed that in the case of the control group that is not treated, the weight did not mostly change, and in the case of Comparative Examples 1 and 10, initially, the weights were slightly changed but were increased again.

However, it may be confirmed that in the case of Examples 1 to 5, a difference in changes of weight loss according to the contents of leptin, adiponectin, and insulin was insignificant, but the weight continuously decreased by 10 to 25% and the decreased weight was mostly maintained.

This is a result confirming that when leptin, adiponectin, and insulin are administered, respectively, the effect of losing weight is insignificant, but when leptin, adiponectin, and insulin are combined and used, the effect of losing weight is more significant.

As the result, it may be confirmed that the combination of leptin, adiponectin, and insulin may generate pharmacologically synergic effect, which may significantly decrease the availability of the ingested nutrient in obese patient requiring a weight control, thereby making it possible to significantly contribute to treatment of obese patients including overweight patients.

The composition according to the present invention has advantages in that side effects by administration of the composition are not generated, and effects of losing weight and then maintaining weight are excellent without changing food intake and consumption amounts, such that the composition may be effectively used to prevent overweight and obesity related diseases.

What is claimed is:

1. A composition for preventing or treating obesity, the composition comprising:
a laptin based polypeptide, which is leptin, leptin derivatives, or a mixture thereof;
an adenosine 5′-monophosphate-activated protein kinase (AMPK) activator; and
insulin, as active ingredients.

2. The composition of claim 1, wherein it comprises 1 to 300 ng/ml of the leptin based polypeptide, 1 to 200 μg/ml of the AMPK activator, and 0.1 to 30 ng/ml of insulin as the active ingredients.

3. The composition of claim 2, wherein it comprises to 250 ng/ml of the leptin based polypeptide, 50 to 150 μg/ml of the AMPK activator, and 1 to 15 ng/ml of insulin as the active ingredients.

4. The composition of claim 1, wherein the AMPK activator is acetyl-CoA carboxylase, malonyl-CoA decarboxylase, or a mixture thereof.

5. The composition of claim 4, wherein the AMPK activator is at least one selected from adiponectin, metformin, phenformin, buformin, thienopyridone, 5′-aminoimidazole-4-carboxamide ribonucleoside (AICAR), resveratrol, nootkatone, and thiazoles.

6. The composition of claim 1, further comprising pharmaceutically acceptable salts.

7. The composition of claim 1, wherein the obesity means that a body mass index (BMI) is more than 25.

8. A health food for preventing or treating obesity, the health food comprising:
a laptin based polypeptide, which is leptin, leptin derivatives, or a mixture thereof;
an adenosine 5′-monophosphate-activated protein kinase (AMPK) activator; and
insulin, as active ingredients.

9. The health food of claim 9, wherein it is at least one kind selected from food, drink, gum, vitamin complexes, health assisting food.
